(19) United States
(12) Patent Application Publication
(10) Pub. No.: US 2023/0263949 A1
(43) Pub. Date: Aug. 24, 2023

(54) Rivet Docking Platform, Occluder

(71) Applicant: NXT Biomedical, LLC, Irvine, CA (US)

(72) Inventors: Joseph Passman, Irvine, CA (US); Alexander Siegel, Irvine, CA (US); Glen Rabito, Irvine, CA (US); Stanton J. Rowe, Irvine, CA (US); Elliot Howard, Irvine, CA (US); Abubaker Khalifa, Irvine, CA (US); Robert C. Taft, Irvine, CA (US)

(73) Assignee: NXT Biomedical, LLC, Irvine, CA (US)

(21) Appl. No.: 18/003,071

(22) PCT Filed: Jun. 25, 2021
(86) PCT No.: PCT/US2021/039218

§ 371 Date: Dec. 22, 2022

Related U.S. Application Data
(60) Provisional application No. 63/046,121, filed on Jun. 30, 2020.

(51) Int. Cl. A61M 1/36; A61B 17/11

(52) U.S. Cl. CPC A61M 1/3655; A61B 17/11; A61B 2017/1107; A61F 2250/0039

(57) ABSTRACT

A variety of methods and devices for manipulating tissue using stents and shunts that foreshorten when expanded. Many of the devices have ends that expand radially more than a center portion of the device, creating an hourglass or rivet shape that is useful for anchoring the device or putting a squeezing force on the tissue.

### RELATED APPLICATIONS

This application claims priority to U.S. Provisional Application Serial No. 63/046,121 filed Jun. 30, 2020 entitled Rivet Docking Platform, Occluder, which is hereby incorporated herein by reference in its entirety.

### BACKGROUND OF THE INVENTION

An artificial shunt creates a hole or provides a small passage that allows movement of fluid from one part of a patient’s body to another, or, more specifically, from one body lumen to another body lumen, one cavity to another cavity, or a combination thereof. Such body lumens can be associated with virtually any organ in the body but are usually associated with lumens in the heart, lungs, cranium and the liver.

Shunts can be used to treat many different conditions. Such conditions include, but are not limited to, pulmonary hypertension, heart failure, hypertension, kidney failure, volume overload, hypertrophic cardiomyopathy, valve regurgitation, and numerous congenital diseases.

Numerous prior art shunt designs exist as exemplified by U.S. Pat. No. 9,510,832, the contents of which is hereby incorporated by reference. As is appreciated by one of skill in the art, the efficacy and safety of a shunt in its intended application largely depends on such attributes as precise shunt placement, secure shunt fixation, shunt durability, minimization of regions of possible fluid stasis, ease of deployment, and adjustability over time, to name a few.

As such, there is a need to constantly improve and refine prior art shunt designs to arrive at a shunt that effectively and safely treats multiple conditions while at the same time allows for ease of use and reduced costs.

### SUMMARY OF THE INVENTION

This application relates to the concepts introduced in U.S. Pat. Application Ser. No. 16/785,501 filed Feb. 7, 2020, and entitled Rivet Shunt And Method Of Deployment, the contents of which are incorporated herein in its entirety. This application introduces shunts that resemble rivets as they have ends that expand like a rivet, preventing the shunt from becoming dislodged from the receiving tissue. The invention disclosed herein includes inventive uses for these rivet shunts, as well as presenting inventive device and methods including, but not limited to docking systems for receiving other devices, stents, and occluders, to name a few.

In one embodiment, the present invention is directed to a shunt that expands to an hourglass shape. As the shunt expands, both of its ends radially flare outwards relative to its middle section. Additionally, the length of the shunt foreshortens which causes the flared ends to engage the tissue surrounding a puncture or aperture within a patient’s tissue, not unlike a rivet. In an alternate embodiment, only one of its ends radially flares outwards relative to its middle section, while the opposite end maintains a diameter similar to its middle section.

In one embodiment, the shunt achieves this shape by having a laser-cut body that forms a plurality of cells. The cells near the middle of the shunt have a smaller size (e.g., length, width) than the remaining cells. The cells near both the proximal and distal ends of the shunt have a larger size (e.g., length, width) than the middle cells, causing them to radially expand to a greater diameter. Further, as the cells radially expand, they increase in width, which causes their length to decrease. The decreased cell length causes the shunt to foreshorten or decrease in length.

In one embodiment, the shunt can be deployed with a balloon catheter. The shunt is compressed over the balloon catheter and, when inflated, causes the shunt to expand.

In one embodiment, the balloon catheter has a balloon that inflates to an hourglass shape. In other words, the balloon’s proximal and distal regions expand to a larger diameter relative to the middle portion.

In one example method of the present invention, a distal end of a balloon catheter has a shunt disposed over its balloon. The shunt and balloon are positioned about halfway through an opening in a patient’s tissue. The balloon is inflated to an hourglass shape, causing the shunt to similarly expand to an hourglass shape while also foreshortening. The flared ends of the shunt are thereby caused to engage the tissue surrounding the opening.

In another embodiment of the present invention, the shunt may include a cover located either along its entire length or along only a portion of its length (e.g., a middle portion).

One embodiment of the present invention includes a method of connecting a circulatory system of a patient to a blood-treatment device comprising: selecting a target vein and an adjacent target artery; deploying a shunt device between the vein and the artery, said shunt device including a non-porous center portion that bridges a gap between the vein and the artery; securing said shunt device in place by flaring opposite ends of said shunt device; and inserting leads to and from the blood-treatment device into a side wall of the non-porous center portion.

In at least one embodiment of this method, inserting leads to and from the blood-treatment device into a sidewall of the center portion comprises inserting needles into the center portion, the needles connected to the leads and in fluid communication therewith.

In at least one embodiment of this method, flaring opposite ends of the shunt device comprises inflating at least one balloon.

In at least one embodiment of this method, the method further comprises removing the leads after a treatment is completed and reinserting leads into the sidewall if a subsequent treatment session is necessary.

Another embodiment of the invention includes a method of improving blood flow to a patient’s heart comprising: harvesting a section of blood vessel from a location in the patient remote from the heart, the section of blood vessel having first and second ends; attaching, ex vivo, a rivet shunt to the first end of the section of blood vessel; attaching, ex vivo, another rivet shunt to the second end of the section of blood vessel; percutaneously delivering one of the rivet shunts to a coronary artery of the patient; inserting a first end portion of the rivet shunt through a sidewall of the coronary artery into an interior lumen of the coronary artery; deploying radial spikes against an inside surface of the sidewall of the coronary artery; deploying radial anchor points against an outside surface of the sidewall opposite the spikes such that the sidewall is sandwiched between the spikes and the anchor points; percutaneously delivering the other rivet shunt to an aorta of the patient; inserting a first end portion of the other rivet shunt through a sidewall of the aorta into an interior lumen of the aorta; deploying radial spikes against an inside surface of the sidewall of the aorta; and, deploying radial anchor points against an outside surface of the sidewall opposite the spikes such that the sidewall is sandwiched between the spikes and the anchor points.

In at least one embodiment of this method, deploying radial spikes comprises inflating a balloon.

In at least one embodiment of this method, deploying radial anchor points comprises inflating a balloon.

In at least one embodiment of this method, said two lumens are separated by a common wall of tissue.

Yet another embodiment of the present invention involves a method of reducing intraocular pressure in an eye of a patient comprising: inserting at least one rivet shunt having an internal lumen through a sclera and a choroid of the eye to create a passage into an aqueous chamber; and, expanding first and second ends of the rivet shunt to anchor the rivet shunt in place.

In at least one embodiment of this method, expanding the first and second ends of the rivet shunt comprises inflating at least one balloon.

One embodiment of the present invention is a rivet shunt that, when implanted in an apex of a heart, provides a repeatably usable access port to an interior of the heart comprising: first and second expandable ends and a center portion defining a center lumen; a valve disposed within the center portion; wherein said valve is displaceable by a tool being passed through the center lumen, allowing the tool access to the interior of the heart; wherein said valve has a high cracking point such that blood is prevented from exiting the heart through the center lumen.

In at least one embodiment of this device, the first and second expandable ends are balloon-expandable.

In at least one embodiment of this device, the first and second expand to a greater than the center portion such that, when the rivet shunt is expanded, the first and second ends have a greater diameter than the center portion.

In at least one embodiment of this device, the rivet shunt foreshortens when expanded, causing the first and second expandable ends to squeeze the tissue therebetween and anchoring the rivet shunt to the apex of the heart.

One aspect of the invention includes a method of manipulating tissue in a patient comprising: inserting at least a first rivet stent in tissue to be manipulated; expanding the first rivet stent, causing ends of the first rivet stent to have larger diameters than a center portion of the first rivet stent, thereby anchoring the first rivet stent in the tissue; and, placing tension on a tether connected to the first rivet stent.

In at least one embodiment of this method, the method further comprises expanding a second rivet stent, causing ends of the second rivet stent to have larger diameters than a center portion of the second rivet stent, thereby anchoring the second rivet stent in tissue spaced apart from the first rivet stent.

In at least one embodiment of this method, placing tension on the tether decreases a space between the first and second rivet stents.

In at least one embodiment of this method, decreasing said space results in a remodeling of a mitral valve.

One aspect of the invention includes a method of improving coaptation of leaflets of a mitral valve comprising: placing an elongated stent in a coronary sinus proximate the mitral valve; and, expanding the elongated stent thereby causing the stent to foreshorten; wherein foreshortening the stent places a squeezing force on tissue adjacent the mitral valve, thereby remodeling the mitral valve and improving coaptation.

In at least one embodiment of this method, expanding the elongated stent comprises inflating a balloon within the stent.

One embodiment provides a device for occluding an opening comprising an expandable braided stent having ends that flare outwardly when said stent is expanded, and a center portion that foreshortens when expanded; a lumen that extends through the braided stent; an elastomeric disc located within said lumen that accommodates balloon expansion and substantially closes when a balloon catheter is removed; wherein when expanded, said ends have a diameter that is greater than an expanded diameter of the center portion.

In one embodiment the small opening is defined by the elastomeric covering.

In another embodiment the small opening comprises a slot formed by two overlapping components of the elastomeric covering.

Another embodiment of the invention is a method of restoring circularity to a misshapen valve annulus comprising inserting a stent into the valve and inflating a balloon within the valve causing the valve to foreshorten while ends of the valve flare radially thereby sandwiching tissue between the ends and anchoring the stent in place. This method may further include inserting a prosthetic valve into the stent.

Yet another embodiment of the invention is a method of occluding a blood vessel comprising: inserting an expandable stent into a blood vessel, the stent having an elastomeric covering on at least one end of the stent capable of blocking blood flow; expanding the stent with a balloon, thereby causing the stent to foreshorten and ends of the stent to flare outwardly, thereby anchoring the stent within the blood vessel.

Still another embodiment is a method of restoring a desired shape to an ostium comprising: selecting a stent having: a first end that, when expanded, flares outwardly to assume desired shape that is sized and shaped such that, when implanted in a targeted ostium, remodels the ostium to have the desired shape; a second end that expands to have a diameter sized to anchor the stent within a vessel leading to or from the ostium; using at least one balloon to expand the stent.

Another embodiment of the invention is a method of joining two tubular body structures end-to-end comprising: surgically implanting an outside stent around adjacent ends of two tubular body structures to be joined; placing an inner stent within the two tubular body structures and aligned with the outside stent; using a balloon catheter to expand the inner stent against the outer stent thereby sandwiching a tissue junction between the inner and outer stents.

Another aspect of the invention is a cerebral-spinal fluid shunt comprising: a first end that flares upon expansion to anchor the shunt into a cerebral-spinal cavity; and a second end that flares proximal the first end and tapers to house a valve that prevents fluid flow from the vein into the cerebral-spinal cavity.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1A is a side view of an embodiment of a rivet shunt of the present invention;

FIG. 1B is a perspective view of an embodiment of a rivet shunt of the present invention;

FIG. 2 is a side view of the shunt of FIGS. 1A and 1B deployed on an expansion device of the present invention in an unexpanded state;

FIG. 3 is a side view of the shunt of FIGS. 1A and 1B deployed on an expansion device of the present invention in an expanded state;

FIG. 4 is a diagram of a first step of an embodiment of a method of forming an A-V shunt of the present invention;

FIG. 5 is a diagram of a second step of an embodiment of a method of forming an A-V shunt of the present invention;

FIG. 6 is a diagram of a third step of an embodiment of a method of forming an A-V shunt of the present invention;

FIG. 7 is a diagram of a fourth step of an embodiment of a method of forming an A-V shunt of the present invention;

FIG. 8 is a diagram of a fifth step of an embodiment of a method of forming an A-V shunt of the present invention;

FIG. 9 is a diagram of a final step of an embodiment of a method of forming an A-V shunt of the present invention;

FIG. 10 is a side view of an embodiment of a CABG connector of the present invention in an unexpanded state;

FIG. 11 is a perspective view of the device of FIG. 10 in an expanded state;

FIG. 12 illustrates a first step in a method of connecting a harvested blood vessel to the device of FIG. 10;

FIG. 13 illustrates a second step in a method of connecting a harvested blood vessel to the device of FIG. 10;

FIG. 14 illustrates a third step in a method of connecting a harvested blood vessel to the device of FIG. 10;

FIG. 15 illustrates a fourth step in a method of connecting a harvested blood vessel to the device of FIG. 10;

FIG. 16 shows a CABG completed using the device of FIG. 10;

FIG. 17 is a detailed cutaway view of the connection between a harvested blood vessel and an aorta;

FIG. 18 is a front view of an eyeball with intra-ocular pressure shunts of the present invention;

FIG. 19. is a side view of an eyeball with intra-ocular pressure shunts of the present invention;

FIG. 20. is a side view of an embodiment of an intraocular pressure shunt of the invention;

FIG. 21 is a cutaway view of an apex of a heart with an embodiment of an access shunt of the invention installed therein;

FIG. 22 is a cutaway side view of the device of FIG. 21;

FIG. 23 is a cutaway view of a step of deploying an embodiment of an LAA occluder of the invention;

FIG. 24 is a cutaway view of a step of deploying an embodiment of an LAA occluder of the invention;

FIG. 25 is a cutaway view of a step of deploying an embodiment of an LAA occluder of the invention;

FIG. 26 is a sideview of an embodiment of a device of the invention;

FIG. 27 is a sideview of an embodiment of a device of the invention;

FIG. 28 is a cutaway view of an embodiment of the invention being used to create a gastrointestinal shunt;

FIG. 29 is a step in a method of using an embodiment of the invention to create a gastrointestinal shunt;

FIG. 30 is a step in a method of using an embodiment of the invention to create a gastrointestinal shunt;

FIG. 31 is a step in a method of using an embodiment of the invention to create a gastrointestinal shunt;

FIG. 32 is a side view of an embodiment of a device of the invention being used to create a CSF shunt;

FIG. 33 is a front view of an embodiment of a device of the invention configured as a closure device;

FIG. 34 is a front view of an embodiment of a device of the invention configured as a closure device;

FIG. 35 is a front view of an embodiment of a device of the invention configured as a closure device;

FIG. 36 is a front view of an embodiment of a device of the invention configured as a closure device;

FIG. 37 is a front view of an embodiment of a device of the invention configured as a closure device;

FIG. 38 is a top view of an embodiment of a device of the invention being used as a tether anchor to remodel a mitral valve;

FIG. 39 is a side view of an embodiment of a device of the invention being used as a tether anchor implanted in an apex of the heart;

FIG. 40 is a top view of a plurality of devices of the invention being used with a tether as an annuloplasty device;

FIG. 41 is a side view of an embodiment of a device of the invention being used with a tether for papillary approximation;

FIG. 42 is a side view of an embodiment of a device of the invention being used with a tether for LV / mitral annulus reshaping;

FIG. 43 is a side view of an embodiment of a device of the invention configured for use as an arterial occluder;

FIG. 44 is a side view of an embodiment of a device of the invention configured for use as a coronary sinus to atrial shunt;

FIG. 45 is a side view of an embodiment of a device of the invention being used in the coronary sinus to reshape a cardiac valve;

FIG. 46 is a side view an embodiment of an inverse rivet device of the invention in an unexpanded state;

FIG. 47 is a side view an embodiment of an inverse rivet device of the invention in an expanded state;

FIG. 48 is a side view of an embodiment of a shaped stent of the invention;

FIG. 49 is a step of a method of the invention for joining two tissue components;

FIG. 50 is a step of a method of the invention for joining two tissue components; and,

FIG. 51 is a step of a method of the invention for joining two tissue components.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The present invention is generally directed to various methods of shunting or occluding body vessels, cavities, appendages, and the like, or a combination thereof. The present invention also applies to devices that make it possible to practice the various shunting methods of the invention.

More specifically, the shunt radially expands to an hourglass or rivet shape while also longitudinally foreshortening. The shunt is initially positioned within a tissue opening and then expanded, which causes the distal and proximal ends of the shunt to flare radially outwards and move towards each other. When fully expanded, these radially flared ends engage the tissue surrounding the opening, creating a smooth transition between either side of the tissue.

This shunt design provides several advantages over prior shunt designs. For example, the shunt may “self-position” within the tissue opening due to its flared shape and therefore provides increased precision in its positioning compared to prior designs. The flared portions also provide strong attachments to the surrounding tissue as compared with prior shunt designs. Finally, the shunt may have a small collapsed profile and yet can expand to a consistent inner diameter with high radial force. This allows the use of low-profile balloons to assist in the expansion of the shunt to achieve consistent and reliable implantation results.

A stent design that can be modified for use as a shunt in accordance with the principles of the present invention as explained herein is disclosed in U.S. Pat. No. 6,068,656 to Oepen, the entire contents of which is incorporated herein by reference.

As discussed in greater detail in this specification, while the foreshortening and hourglass shape of the various rivet designs of the present invention make the methods disclosed herein possible, this shape can be achieved in several different ways and the shunts themselves may have several different features. Moreover, though the methods herein will be associated with a rivet device that is likely best suited for the particular method being discussed, it should be explicitly understood that other devices discussed herein and in the incorporated references may be substituted without changing the scope of the methods.

### A-V Fistula Creation in CKD Patients

Hemodialysis patients often undergo arteriovenous (A-V) fistula formation for creation of durable vascular access. This fistula creates a larger opening with less resistance to fluid flow than the natural path from artery to vein through the capillaries. The increased fluid flow is necessary to shorten the length of the dialysis procedure and to protect the delicate distal vasculature from damage due to higher-than-normal pressures encountered during dialysis.

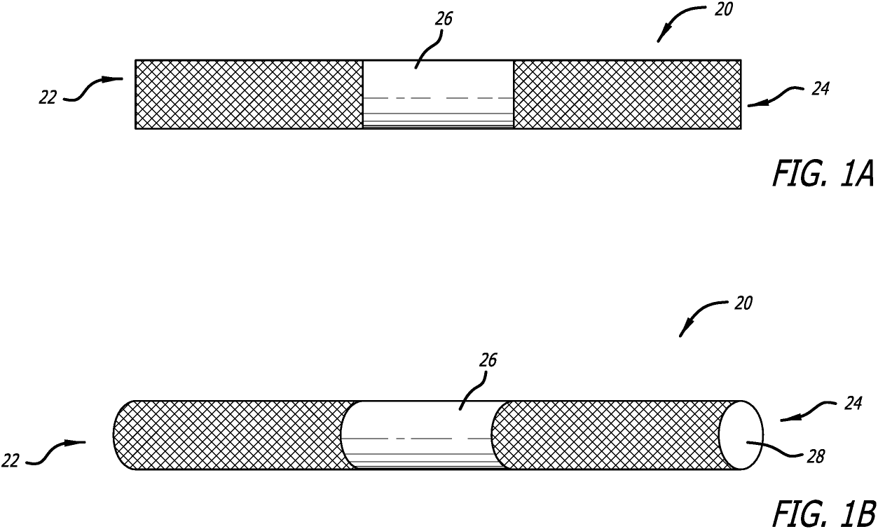

The preferred approach involves the creation of a radial to brachial fistula, which is a distal to proximal ideology. The radial access is not often used with non-surgical techniques because a large tissue gap exists between the artery and vein. This problem is overcome using the method and device shown in FIGS. 1-9, which allow an interventional solution that could enable repeatable and durable interventional creation of a distal A-V fistula.

FIGS. 1A and 1B show a rivet device 20 having first and second end portions, 22 and 24, and a non-porous center portion 26. The end portions 22 and 24 flare or expand radially when deployed to provide anchoring to the device 20. The ends 22 and 24 may self-expand, or they may be mechanically expanded, such as by balloons or other expansion devices.

The center portion 26 has a central lumen 28 (see FIG. 1B) that serves as a fluid passage or conduit between the two ends. The center portion 26 is constructed of a non-porous solid material capable of carrying fluid without leaking. The center portion 26 is preferably rigid or semi-rigid. In at least one embodiment, the center portion 26 is formed of a material capable of accepting a needle such that it may provide an access point for an external conduit, such as catheters leading to and from a dialysis machine. This access point prevents damage to vascular access points through repeated connections to a dialysis machine that arise from multiple, successive dialysis sessions.

The center portion 26 is sized to span the gap between an artery and an adjacent vein at a given target site. The lumen 28 of the center portion 26 is similarly sized to accommodate the flow rate at a given target site. Alternatively, the lumen 28 may be sized to provide a desired, yet restricted, flow depending on the target site and the desired effect of the rivet shunt.

Examples of materials usable for the center portion 26 include, but are not limited to PTFE/ePTFE, polyurethane, silicone, and the like. Examples of materials for use in creating the end portions 22 and 24 include, but are not limited to, nitinol, stainless steel, and biodegradable materials like PLGA, magnesium, etc. The end portions 22 and 24 may be braided or woven wires, fenestrated or laser-cut tubing, or other acceptable expandable constructions.

The material and/or construction of one of the end portions 22 and 24 may or may not be different than the other end portion. Similarly, the material used for the center portion 26 may or may not be different than that of the end portions. In one embodiment, the entire device is cut from a tube, the ends being fenestrated for purposes of expansion and the center portion 26 remaining solid. In at least one embodiment the device 20 includes a continuous length of braided or fenestrated tubing. The center portion 26 further includes a length of tubing placed around a middle of the braided or fenestrated length of tubing and bonded thereto. Alternatively, in at least another embodiment, the device 20 may include a continuous length of braided or fenestrated tubing with the center portion 26 further including a length of tubing bonded to an inside surface of the length of braided or fenestrated tubing. In at least one other embodiment, the device 20 may include a continuous length of braided or fenestrated tubing with a center portion 26 that has a non-porous material applied to one or both sides of the continuous length of tubing, perhaps embedding the center portion of the braided or fenestrated tubing in non-porous material. The non-porous material would preferably prevent the center portion 26 from expanding. In still other embodiments, one or both end portions 22, 24 are a different material than the center portion 26. The end portions are then bonded, welded or otherwise connected to the center portion 26.

In one example, when compressed, the rivet shunt 20 has a length of about 20 mm and a diameter of about 1.5 mm, and when expanded, the end portions 22 and 24 of the rivet shunt 20 have a diameter of about 8 mm. The center portion may have a diameter of about 5 mm.

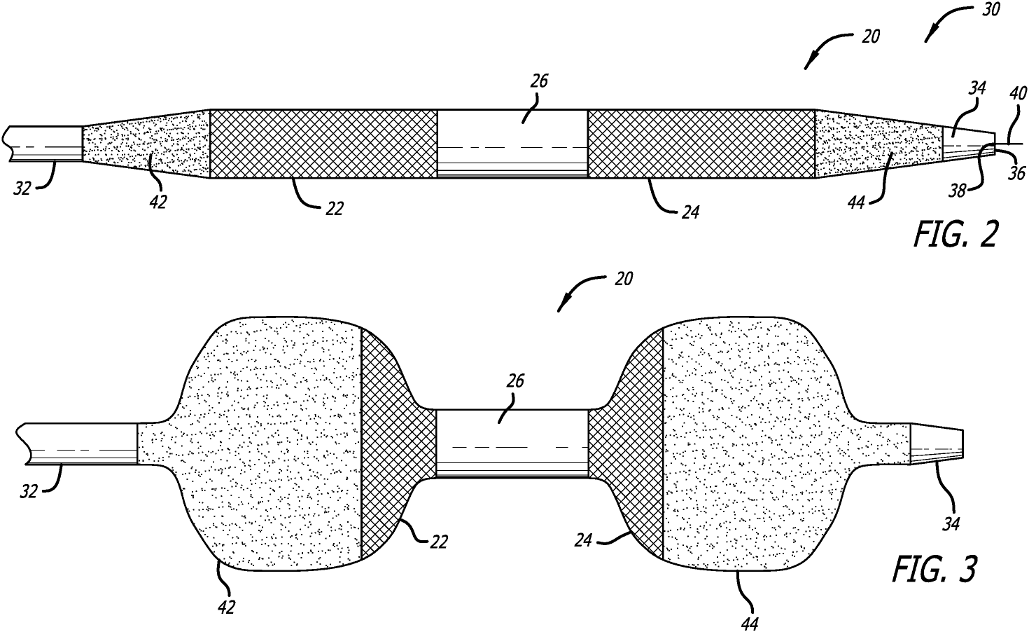

Referring to FIGS. 2-3, there is shown an embodiment of an expansion device 30 usable for implanting the rivet shunt 20. The expansion device 30 is a balloon catheter including a catheter 32 terminating in a nosecone 34 at a distal end 36 thereof. The catheter 32 may have a continuous guidewire lumen 38 to accommodate a guidewire 40. The guidewire 40 may have a sharpened tip or is preferably a radiofrequency (RF) guidewire. The RF energy is useable to puncturing and ablating/sealing the puncture sites.

The expansion device 30 further includes a proximal balloon 42 and a distal balloon 44 usable to expand the end portions 22, 24. Alternatively, the device 30 utilizes a single, elongate balloon, that is longer than the center portion 26. During expansion, the non-expandable, or less-expandable center portion 26 ensures that the end portions 22, 24 expand more than the center portion 26, giving the expanded device 20 a rivet shape such that the end portions 22, 24 anchor the device 20. In one example, the balloon 44 can be composed of a compliant material and a non-compliant band (not shown) can be positioned around the balloon 44 corresponding to the center portion 26 of the shunt 20. In another example, the balloon 44 may be constructed such that a proximal region 44A and a distal region 44B can be composed of a material with different expansion properties than a middle region located within the center portion 26 of the device 20. FIG. 2 shows the device in a compressed configuration and FIG. 3 shows the device in an expanded configuration.

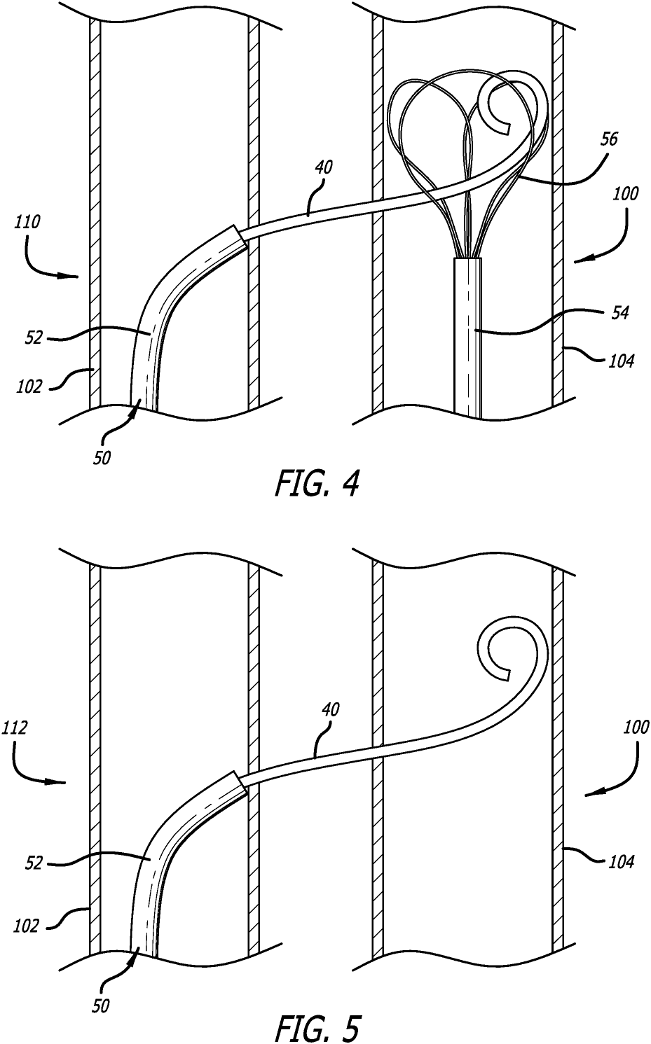

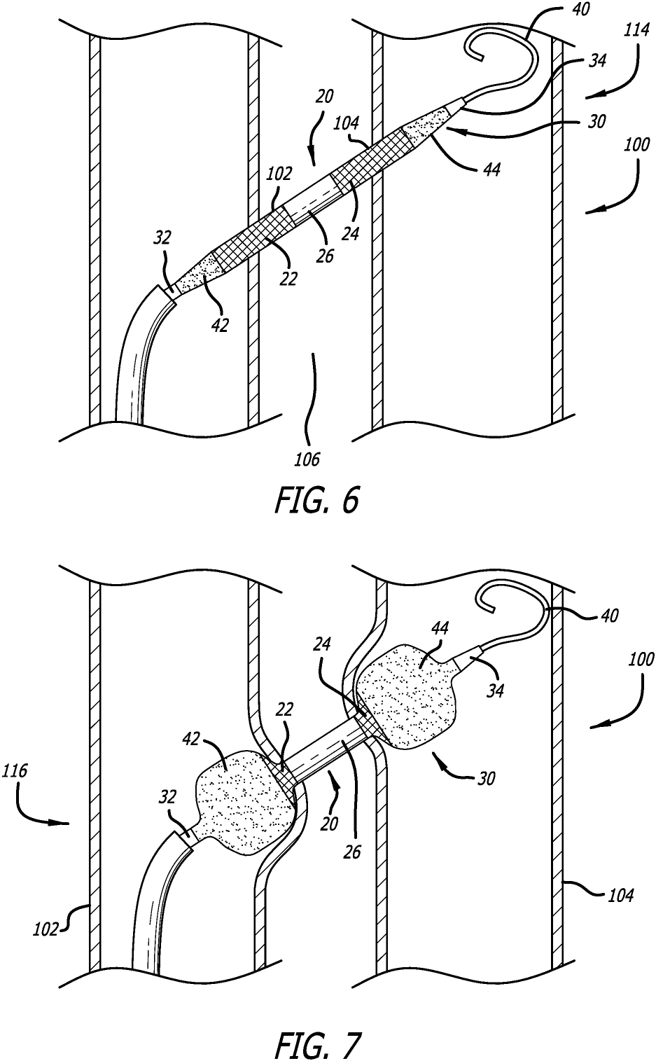

An implantation method 100 for creating a shunt between a first location 102 and a second location 104 using the device 20 is illustrated in FIGS. 4-9. FIG. 4 shows the first step 110 of the method 100, which involves placing a delivery device 50, comprising a crossing sheath 52 containing the RF guidewire 40, expansion device 30 (FIG. 6) and implant 20 (FIG. 6), at the first location 102, preferably a vein as opposed to an artery to limit the amount of time the artery is punctured. A target sheath 54 with a radiopaque target snare or balloon 56 is navigated to the second location 104 to receive the RF guidewire 40.

FIG. 5 shows a next step 112 of the method 100 in which the RF wire 40 has successfully punctured a wall of the target artery at the second location 104 such that the distal end of the wire 40 is contained within the artery. The target sheath 54 may be removed at this step.

FIG. 6 shows a next step 114 of the method 100. At 114, the implant 20 and expansion device 30 are advanced over the guidewire 40 until the center portion 26 is centered in a tissue gap 106 between the first and second target locations 102 and 104 using an imaging modality such as serial angiography, for example.

FIG. 7 shows a next step 116 of the method 100 in which the balloon or balloons 42 and 44 are inflated to expand the end portions 22 and 24. Expanding the device 20 causes the device 20 to foreshorten, drawing the vein and the artery closer together, and helping to seal the target locations 102 and 104 against the center portion 26, as shown.

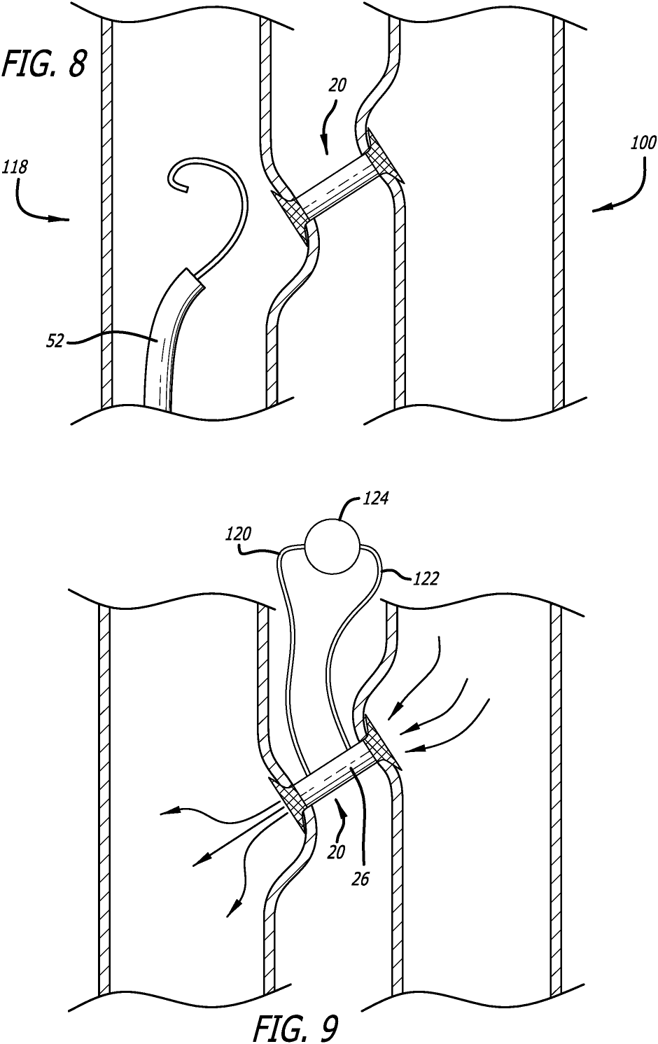

FIG. 8 shows the final step 118 of the implantation method 100. Step 118 involves retracting the guidewire into the expansion device 32 (not shown) and retracting the expansion device 32 (not shown) into the crossing sheath 52. These two retraction actions can be completed sequentially or simultaneously. FIG. 9 shows the operational rivet shunt 20 establishing blood flow from the artery to the vein across the tissue gap 106. Also shown are catheters 120 and 122 attached to the center portion 26 and leading to and from a dialysis machine 124. If the center portion 26 is to be used as a connection point to the dialysis machine 124, as shown, the center portion 26 may be radiopaque and/or magnetic to assist in locating the center portion 26 when attaching the lead catheters 120 and 122.

In one example, a rivet shunt 20 designed for use with the dialysis machine, has a compressed length of less than 20 mm, a diameter of less than 1.5 mm, and when expanded, the end portions 22 and 24 of the rivet shunt 20 have a diameter of less than 8 mm, preferably about 4 mm. The center portion may have a diameter of less than about 5 mm, preferably about 2 mm.

### CABG Attachment

Coronary artery bypass grafting (CABG), also known as heart bypass surgery is a procedure to improve poor blood flow to the heart caused by conditions such as obstructive coronary artery disease, a type of ischemic heart disease. CABG may also be used in an emergency, such as a severe heart attack, to reestablish blood flow. An example of an existing device used for CABG is the P.A.S.Port device made by Cardica, Inc.

CABG uses blood vessels from another part of the body and connects them to blood vessels above and below the narrowed artery, bypassing the narrowed or blocked coronary arteries. One or more blood vessels may be used, depending on the severity and number of blockages. The harvested blood vessels are usually arteries from the arm or chest, or veins from the legs. Synthetic vessels may also be used.

Risks and possible complications may occur with this procedure. After CABG, a patient may require medicines and heart-healthy lifestyle changes to further reduce symptoms and help prevent complications such as blood clots. Typical CABG procedures are surgical and extremely invasive.

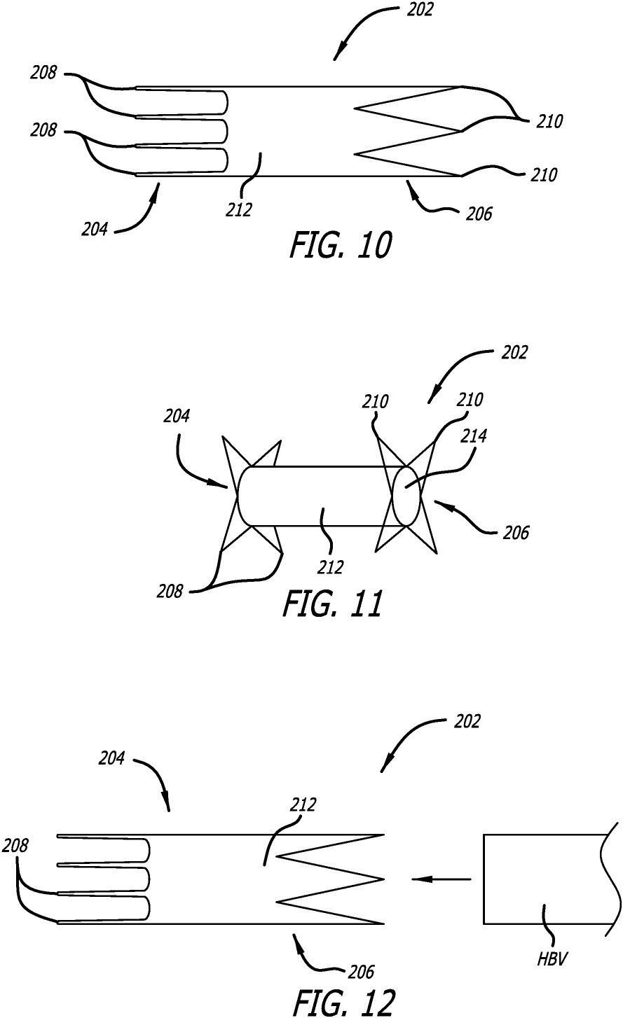

FIGS. 10-17 show a CABG method 200 and a rivet shunt device 202 to connect a coronary artery CA to an aorta A. The rivet shunt 202 is designed to be attached to either end of a harvested blood vessel ex vivo. Referring to FIG. 10, the rivet shunt 202 has a first end 204 and a second end 206. The first end 204 is characterized by a plurality of sharp, narrow spikes 208 that can be used to puncture tissue. The second end 206 is characterized by long anchor points 210. The body 212 of the rivet shunt 202 may be braided, fenestrated or solid. In some embodiments, the body 212 is not expandable and has an inside diameter that closely matches, or is slightly larger than, the outside diameter of the harvested vessel. The first end 204 and second end 206 expand outwardly and fold back to assume the configuration shown in FIG. 11. In another embodiment, the body 212 also expands and foreshortens significantly, thus aiding in the anchoring of the device, as explained further below.

The ex vivo construction process is shown in FIGS. 12-14. FIG. 12 shows a harvested blood vessel HBV being passed through an internal lumen 214 of an unexpanded rivet shunt 202. Once an end of the HBV emerges from the first end 204 of the rivet shunt 202 and past the spikes 208, the spikes are bent inwardly against the tissue of the HBV, as shown in FIG. 13.

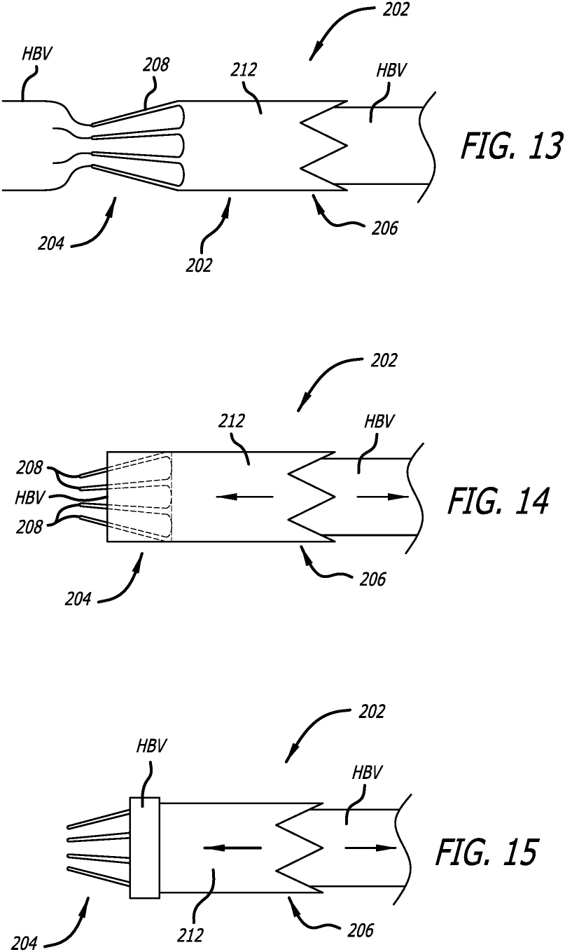

Next, as seen in FIG. 14, the HBV is pulled, or the rivet shunt 202 is advanced, (see arrows) causing the spikes 208 to puncture the tissue of the HBV. Once the resulting punctures are located at a base of the spikes 208, where the spikes 208 meet the body 212, the tissue may be folded rearwardly, as shown in FIG. 15. This process is then repeated on the other side of the HBV with a second rivet shunt 202.

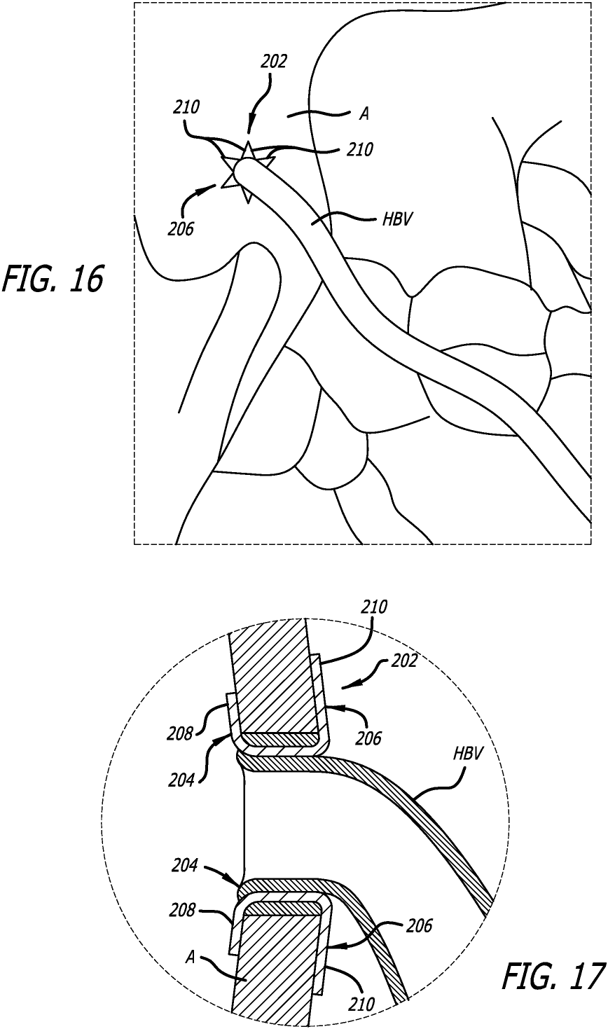

FIG. 16 shows the shunt 202 being used in a CABG procedure to connect an HBV to an aorta A. The anchor points 210 of the second end portion 206 are visible on the outside surface of the aorta. FIG. 17 shows a cutaway side view of the connection between the aorta A and the HBV using the shunt 202. The first end portion 204 is located on an inside surface of the aorta A. The spikes 208 are deployed radially against the inside wall of the aorta. The second end portion 206 is located on the outside wall of the aorta A and the anchor points 210 are deployed radially against the outside wall such that the wall is compressed between the first end 204 and the second end 206. The end of the HBV is folded around the first end 204 and secured in place.

### Reduction of Intraocular Pressure (IOP)

Abnormally high intraocular pressure (IOP) can result in damage to the optic nerve, a condition known as glaucoma. Glaucoma is the leading cause of blindness for people over the age of 60. Damage to the optic nerve can be avoided by relieving fluid pressure from the aqueous or anterior chamber, which is filled with a fluid called aqueous humor. Attempts have been made at implanting devices through the sclera and choroid to relieve excessive pressure. One example of such an effort is the Baerveldt shunt. Another example is the Ahmed shunt. The difference between the two is that the Ahmed shunt is valved and the Baerveldt shunt is non-valved. Inserting either of the two shunts requires blunt dissection of the cornea and is sutured in place. The rivet shunts described herein are less-invasively implanted and do not require sutures.

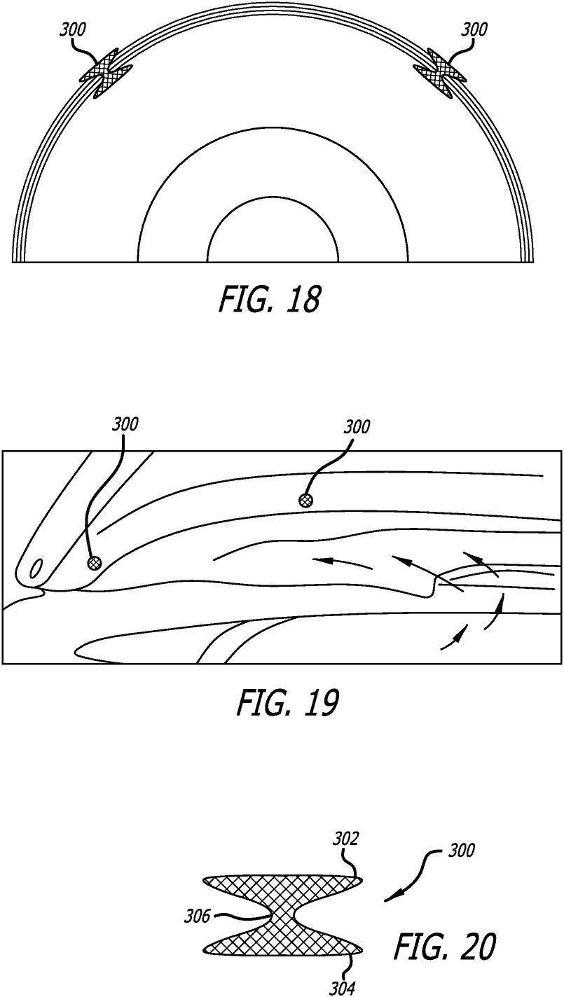

FIGS. 18-20 show a device and a method for reducing intraocular pressure (IOP) in adult patients with mild-to-moderate primary open-angle glaucoma. The method involves placing one or more rivet shunts 300 through the sclera and choroid to create a passage into the aqueous chamber. The shunts 300 allow fluid pressure in the aqueous chamber to be relieved through the shunt and into the eye socket where the fluid can flow around the eyeball and get pumped out of the eye through the puncta.

FIG. 18 is a front view of the eye showing a pair of shunts 300 located at approximately the 10:30 and 1:30 clock positions around the eye. FIG. 19 is a side cutaway view of the eye showing alternate positioning of the shunts 300 in which one of the shunts is located closer to the front of the eyeball and the other shunt is located behind it. One skilled in the art will understand that more than two shunts could be used to allow the release of more fluid. Additional shunts may be preferable to increase fluid flow, as opposed to using larger shunts, due to the sensitivity of the eye.

FIG. 20 shows an example of a shunt design 300 usable to reduce intraocular pressure according to the method described above. The shunt 300, when expanded, has an hourglass shape with flared ends 302 and 304 and a narrow center portion 306. The shunt 300 may be fenestrated or braided and may be a miniature version of any of the other shunts described herein.

### Vascular/Chamber Access/Closure

Certain cardiac procedures require repeated access through the muscle wall of the heart. For example, one access point used to access the aortic valve, or the mitral valve involves penetrating the apex of the heart. Repeatedly puncturing the heart can create unnecessary trauma to the muscle wall.

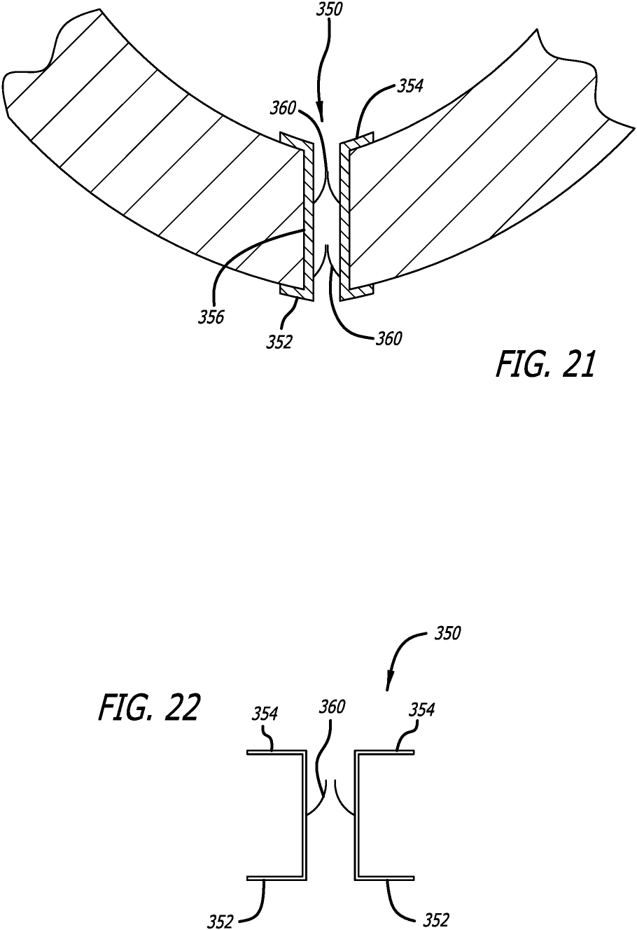

FIGS. 21 and 22 show a rivet shunt device 350 that, like other embodiments described herein, has radially expanding ends 352 and 354 that serve as anchors, and a center portion 356 that creates a lumen or passage 358 through the device 350. Additionally, the device 350 also includes a valve or sealing mechanism 360. The sealing mechanism 360 may include one or more valves designed to be easily displaced by a tool being introduced into the heart through the lumen 358. The sealing mechanism 360 is designed with a high “cracking” point, meaning that the mechanism 360 is able to withstand the significant pressures created by the ventricles without leaking or otherwise failing.

FIG. 21 shows an embodiment of the shunt 350 having two sets of valves 360 to increase resistance to flow and increase sealing power. The shunt 350 of FIG. 21 is depicted as being installed in the apex of a heart. FIG. 22 shows an embodiment of the shunt 350 having a single valve 360. The sealing mechanisms 360 are depicted as duckbill valves but one skilled in the art will realize that other high pressure check valve designs may be substituted as long as they allow access through the shunt for a tool or catheter. Once installed, the shunt device 350 allows repeated access through the shunt 350 with a tool or catheter.

### Left Atrial Appendage (LAA) Occlusion

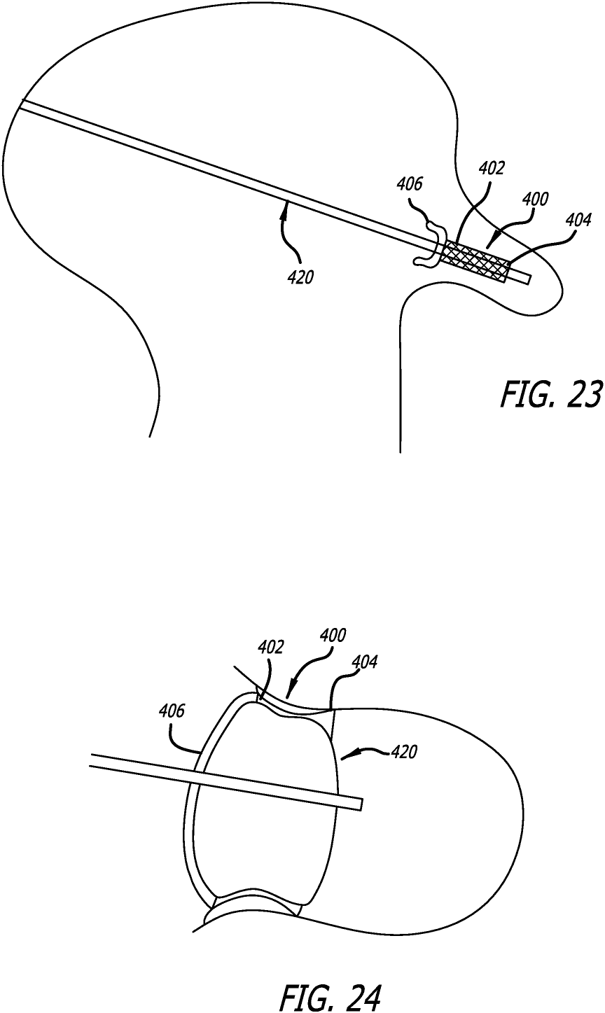

FIGS. 23-25 show the use of the rivet design of the present invention as an LAA occluder. The LAA occlusion implant includes a rivet stent 400 that is expandable against the entrance walls to the LAA. The stent 400 includes ends 402 and 404 that flare outwardly when expanded. Furthermore, end 402 has an elastomeric covering 406 that, when the stent is expanded, becomes somewhat taut and prevents or restricts fluid from flowing into the LAA. In some embodiments the covering 406 is a complete covering. In other embodiments the covering 406 includes an aperture 408 that allows the balloon catheter to be removed and which closes down to prevent flow after removal. In yet another embodiment, the stent 400 has no elastomeric covering and is used as a dock for an existing LAA occluding device, such as the Boston Scientific Watchman device.

FIG. 23 shows a first step in implanting the rivet stent 400. The stent 400 is placed over a balloon catheter 420 and introduced to the opening of the LAA using a transseptal approach.

Next, as shown in FIG. 24, the balloon catheter 420 is inflated, expanding the stent 400 and causing ends 402 and 404 to flare, thereby pinching tissue of the LAA and anchoring the device 400 in place. Expanding the stent 400 further causes the covering 406 to become taut.

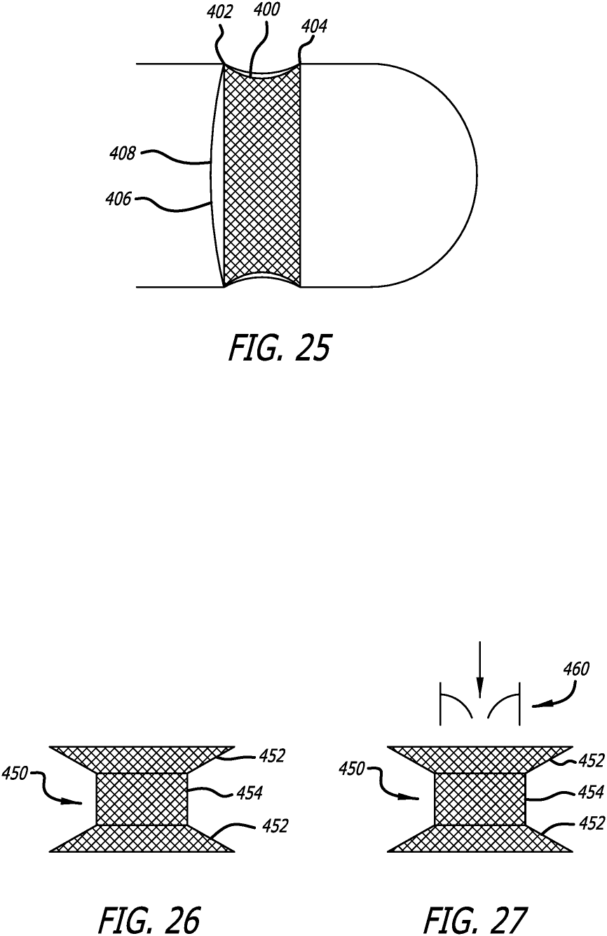

Finally, as seen in FIG. 25, the balloon catheter is deflated and removed, leaving the stent 400 in place and allowing the covering 406 to limit or block fluid from flowing into the LAA.

### Valve Dock

FIGS. 26 and 27 show a rivet stent 450 of the invention being used as an ideal surface (dock) for receiving a prosthetic valve 460. The rivet stent 450 has ends 452 that flare outwardly when the stent 450 is expanded. A center portion 454 has an inner lumen 456 of a relatively constant diameter or uniform surface that ensures a prosthetic valve 460 expanded or otherwise placed into the inner lumen 456 is optimally suited, thus preventing paravalvular leakage. FIG. 27 show a valve 460 being inserted into the expanded stent 450. One example of an application is a docking platform for a trans-aortic valve repair (TAVR) device. The rivet stent 450 may be navigated to an aortic valve and expanded within the aortic valve. In one embodiment, the leaflets are excised prior to deployment. In another embodiment, the leaflets are pushed out of the way by the rivet stent 450 during balloon expansion. The balloon is then deflated and a TAVR implant is then deployed within the rivet stent 450. In another embodiment, the rivet stent 450 is deployed as a repair device to restore circularity to the annulus of the aortic valve.

In another embodiment, the rivet stent 450 is used as a docking platform for a trans-mitral valve repair (TMVR) device. The rivet stent 450 may be navigated to a mitral valve and expanded within the mitral valve.. In one embodiment, the leaflets are pushed out of the way by the rivet stent 450 during balloon expansion. The balloon is then deflated and a TMVR implant is then deployed within the rivet stent 450. In another embodiment, the rivet stent 450 is deployed as a repair device to restore circularity to the annulus of the mitral valve.

### Gastro-Anastomosis

One or more of the rivet shunts of the present invention could be used to form an anastomosis in the gastrointestinal tract. Doing so creates a bypass that diverts some or all of the nutrients traveling through the digestive tract through the anastomosis instead of following the natural path. The bypass may be used to treat conditions such as obesity and type II diabetes.

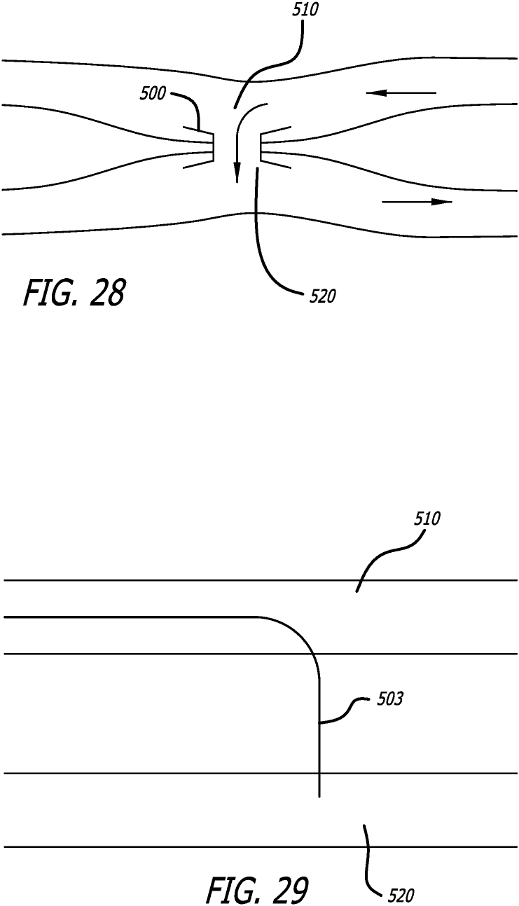

FIG. 28 shows a rivet shunt 500 creating an anastomosis between two locations 510 and 520 of the small intestine. The horizontal arrows in the intestines show the natural flow path while the vertical arrow through the shunt shows the bypass path. The shunt is implanted by passing a guidewire 503 from a target location in one portion of the intestine, through the intestinal walls to a target location in a second intestine, as shown in FIG. 29.

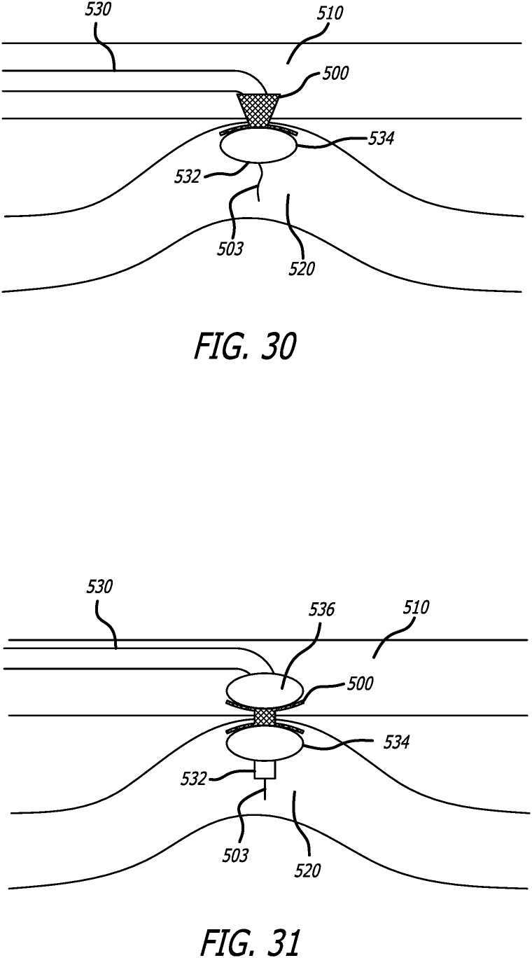

As seen in FIG. 30, a balloon catheter 530 carrying the shunt 500 is then advanced until a distal end 532 reaches the second location 520. A distal balloon 534 is then inflated, expanding a distal end 502 of the shunt 500. The catheter 530 is then retracted with the distal balloon 534 still inflated in order to reduce the gap between the two locations 510 and 520.

Next, as shown in FIG. 31, a proximal balloon 536 is inflated, foreshortening the shunt 500, flaring a proximal end 504, and compressing the two locations 510 and 520 together. The catheter 530 may then be deflated and removed, leaving the configuration shown in FIG. 28.

### Cerebral-Spinal Fluid (CSF) Shunt

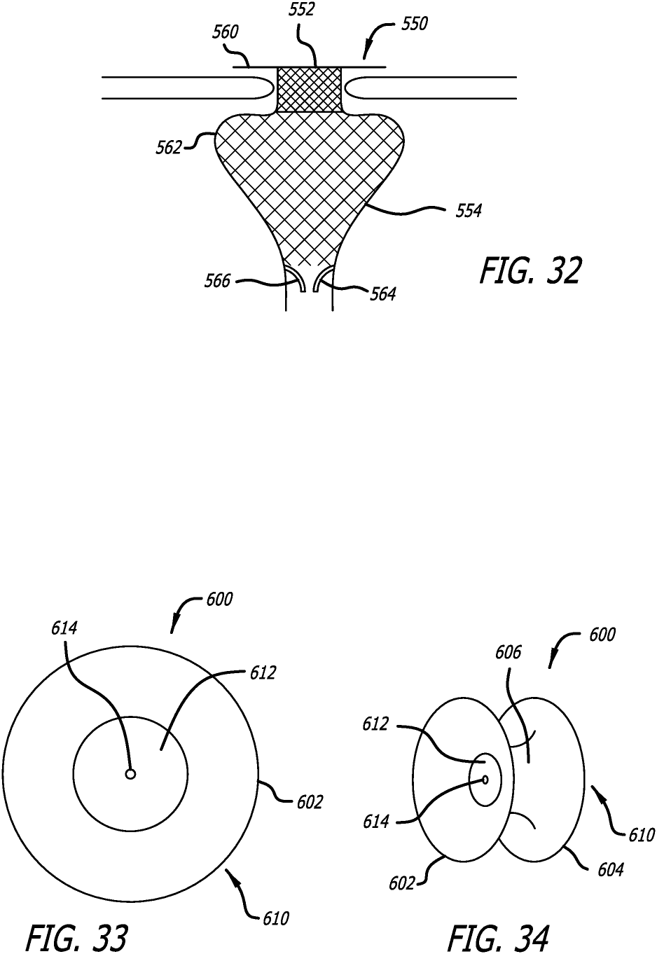

FIG. 32 shows a shunt 550 of the invention being used as a CSF shunt in order to relieve pressure caused by CSF. The shunt creates a fluid path between a cerebral-spinal cavity 552 into a vein 554 in order to relieve pressure. The shunt 550 has a flared first end 560 and a flared second end 562 that may taper into a passage 564 containing a valve 566. Alternatively, the valve dock concept described above may be used for this purpose.

### Closure Devices

FIGS. 33-37 show various closure devices that may be used to treat defects such as atrial septal defects (ASD), patent foramen ovale (PFO), ventricular septal defects (VSD) and others. Generally, each of the devices includes a rivet stent 600 that, like the others described herein, foreshortens and has ends 602 and 604 that flare when the stent is expanded. The stent 600 also has a central portion 606 between the ends 602 and 604 that is narrowed in relation to the ends 602 and 604.

FIGS. 33 and 34 show a device 610 that uses the stent 600 and has an elastomeric disc 612 in the central portion 606. The disc 612 includes a pinhole 614 in the center that stretches to accommodate a balloon catheter but closes when the catheter is removed to close the central portion 606.

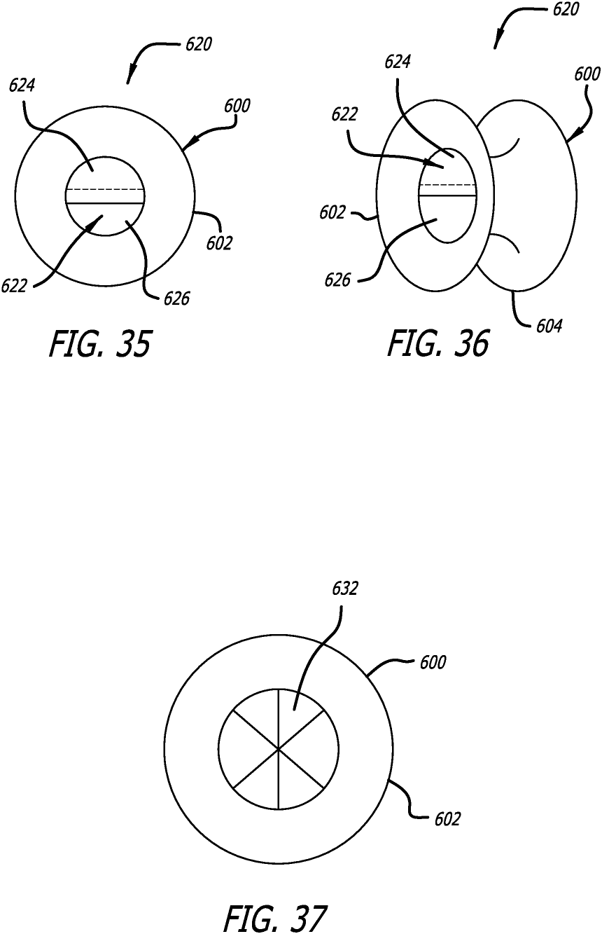

FIGS. 35 and 36 show a device 620 that is like device 610 in that the stent 600 may be the same but the elastomeric occluder 622 includes first and second overlapping layers 624 and 626 that are semi-circular such that a balloon catheter may be threaded between the two layers for expansion. After removal, the layers overlap to block flow through the device 620.

FIG. 37 shows a device 630 that has a stent 600 with an occluder 632 having an iris design like a camera shutter. The occluder can open to accommodate a balloon catheter and closes after the catheter is removed to prevent fluid flow through the device 630.

### Anchors

There are many medical uses for tethers. The versatility of medical tethers is analogous to the versatilities of ropes. They can be used in a great multitude of situations where it is desired to bring one organ or tissue closer to another, or to prevent unwanted shifting of an anatomical feature that lacks the ability to prevent migration.

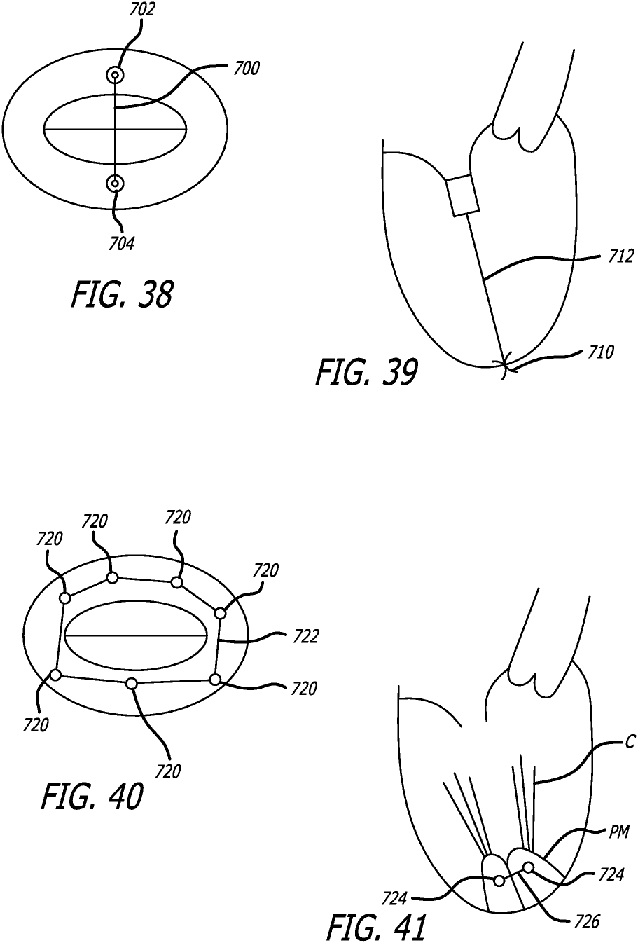

Referring to FIG. 38, there is shown an application for a rivet stent of the present invention to be used as annular anchors in an annuloplasty procedure. The example shown in FIG. 38 is a tether 700 anchored to opposite sides, anterior and posterior, of a mitral valve and tensioned in order to bring the opposite sides closer together to reestablish coaptation of the two leaflets of the mitral valve. The tether 700 is connected at either end to first and second rivet anchors 702 and 704, which are attached to the annulus of the mitral valve. The anchors 702 and 704 are constructed according to any of the rivet devices described herein but are preferably a closed design that does not include an open central lumen.

FIG. 39 shows another application in which the rivet stent is used as an anchor. A rivet anchor 710 is placed near the apex of a ventricle. A tether 712 is attached to the anchor and is secured at an opposite end to a coaptation device implanted in the mitral valve. An example of such a device is the Forma device by Edwards Lifesciences. The anchor would help prevent migration of the coaptation device during contraction of the left ventricle.

Alternatively, as seen in FIG. 40, several rivet anchors 720 could be placed around a valve annulus and connected with a tether 722. Tightening the tether creates an annuloplasty device that could be implanted non-surgically using a catheter. Other transcatheter annuloplasty devices are more complicated and customizable for a given valve geometry. One example of a transcatheter annuloplasty device is the Boston Scientific Millipede device.

FIG. 41 shows papillary approximation application for the rivet anchor/tether combination of the present invention. In FIG. 41 and one or more rivets 724 are placed in the left and right papillary muscles in the left ventricle and one or more tethers 726 are routed between the rivet anchors. Tightening the tether pulls papillary muscles PM together, in turn pulling the chordae C together and improving mitral coaptation.

FIG. 42 shows a rivet anchor 730 and a tether 732 being used for LV / mitral annulus reshaping. An end 734 of the tether 732 is attached to a distal end of a probe 736. An opposite end 738 of the tether 730 is connected to the rivet anchor 730, shown as being implanted by a delivery catheter 740.

### Arterial Occluder

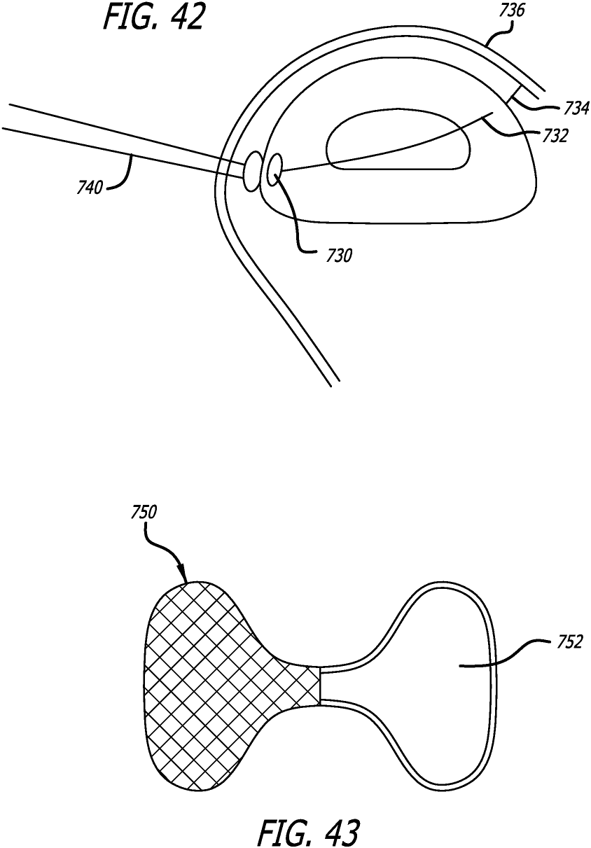

FIG. 43 shows an embodiment of a rivet stent 750 configured for use as an arterial occluder to stop blood supply to tumors, etc. The rivet 750 has an hourglass shape and has flow-resisting covering 752 at one or both ends. The diameter of the ends is greater than that of the artery to prevent migration. When deployed, the rivet stent 750 stops blood flow through a targeted artery. An example of a prior art device is the Medtronic MVP device.

### Coronary Sinus Applications

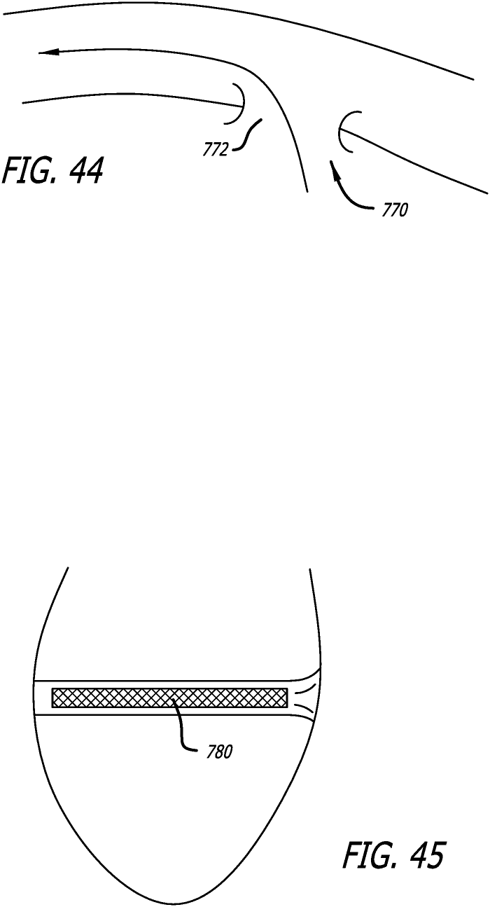

FIG. 44 shows a rivet stent 770 being used as a coronary sinus to atrial shunt. The rivet stent 770 may be any of the open stent designs described herein. A stent covering 772 is used to prevent leakage. The coronary sinus is a large vein that runs along the posterior of the heart and collects blood from several myocardial veins and delivers the blood to the left atrium. In certain circumstances, there is a need to create a shunt between the coronary sinus and the left atrium. This shunt does not span any gaps as the coronary sinus is attached to, and runs along, the heart wall surrounding the left atrium. as shown in FIG. 44. Shunt 770 thus provides an attractive alternative to more complicated devices such as the Edwards Atrial shunt.

FIG. 45 shows an elongated rivet stent 780 implanted in the coronary sinus. The stent foreshortens significantly during expansion. If the stent 780 has an unexpanded diameter that is close to that of the coronary sinus, the stent 780 will engage the tissue of the coronary sinus well before reaching a maximum expansion. Once tissue is engaged, the stent will place a pulling force on the coronary sinus during foreshortening. Because the coronary sinus runs along the heart wall, the pulling force will be transferred to the heart wall. This transference can be used to reshape the mitral valve to establish coaptation, while keeping the mitral valve completely isolated from the implant. Using an elongated rivet stent 780 in this way may provide advantages over other devices, such as the Edwards Monarc device, because the foreshortening is controlled during implantation, allowing an optimal reshaping of the mitral valve.

### Shaped Applications

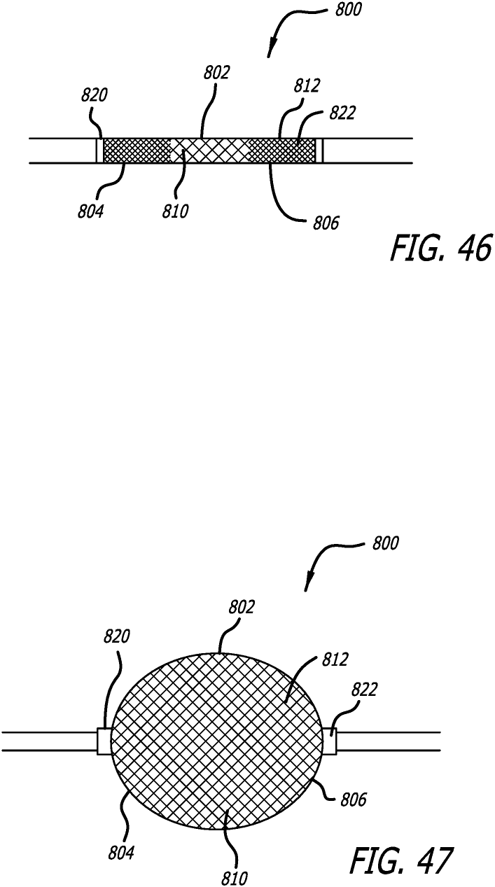

The variability of the braided construction of the devices discussed herein lends these device to a variety of other applications. For example, FIGS. 46 and 47 provide an “inverse rivet” design 800 in which a midportion 802 expands more than the ends 804 and 806. This can be accomplished by providing cells 810 in the midportion 802 that are larger than cells 812 near the ends. After expansion with a balloon, the inverse rivet 800 takes on a spherical shape. The ends 804 and 806 may additionally be restrained with bands 820 and 822 to inhibit the ends 804 and 806 from expanding, further ensuring a spherical expansion shape.

The inverse rivet 800 may have many applications. For example, the device 800 may be used for embolization. In this regard, the rivet 800 could include a coating such as a drug-eluting coating or a tissue swelling coating. The rivet 800 could be sized for implantation as an LAA occluder.

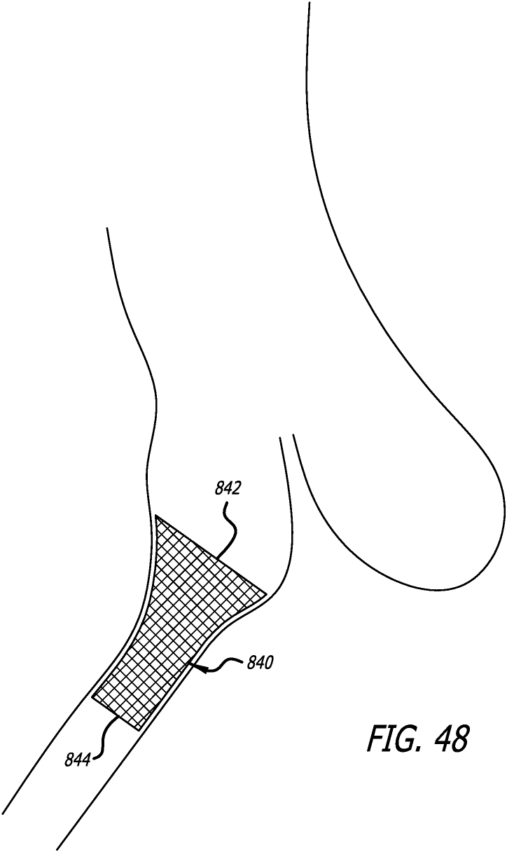

Another example is shown in FIG. 48. A shaped stent 840 has one end 842 that is flared and a second end 844 that is not flared. The flared end 842 is sized and shaped to match a desired shape for an ostium to the coronary artery and is thus usable to optimize circulation through the coronary artery.

### Tubular Connectors Using Multiple Stents

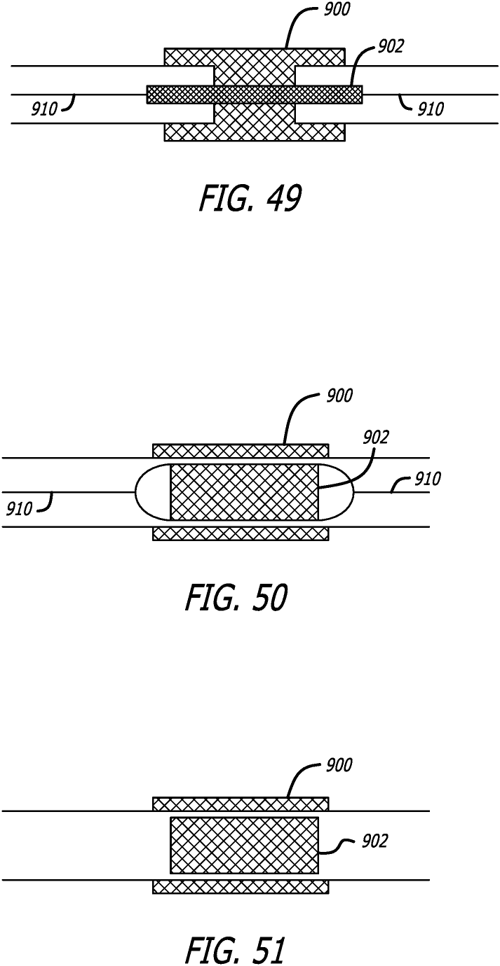

FIGS. 49-51 show an application usable to join two tissue components, such as vessels or other structures, together. In this application, an outside stent 900 is used in combination with an inside stent 902. The outside stent 900 is surgically placed around the outside of a tissue structure such as at the junction of two vessels to be joined. A second stent 902 is placed within the vessels and aligned with the outside stent. This is shown in FIG. 49. In FIG. 50, a balloon catheter 910 is used to expand the inner stent 902 against the outer stent 900, thereby sandwiching the tissue junction between the two stents 900 and 902. The foreshortening of the stents 900 and 902 during expansion brings the two vessels closer together, preventing leaks. At FIG. 51, the balloon catheter is deflated and removed.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1-36. (canceled)

37. A method of creating a fistula in a patient, comprising:
placing a delivery device into an artery and a vein of a patient;
radially flaring a first end of a shunt within the artery and against a wall of the artery, and radially expanding a second end of the shunt within the vein and against a wall of the vein, while also longitudinally foreshortening the shunt; and,
establishing blood flow from the artery to the vein, through a covered center portion of the shunt and across a tissue gap.

38. The method of claim 37, further comprising locating the covered center portion of the shunt and attaching a dialysis machine to the covered center portion of the shunt.

39. The method of claim 38, wherein the covered center portion is connected to a first dialysis catheter and a second dialysis catheter.

40. The method of claim 39, wherein the covered center portion may be radiopaque or magnetic to assist in locating the covered center portion.

41. The method of claim 37, wherein radially flaring the first end of the shunt and radially flaring the second end of the shunt comprises allowing the first end and the second end of the shunt to self-expand.

42. The method of claim 37, wherein radially flaring the first end of the shunt and radially flaring the second end of the shunt comprises inflating a balloon catheter.

43. The method of claim 42, wherein the inflating the balloon catheter further comprises inflating a proximal balloon and a distal balloon to radially flare the flare the first end and the second end of the shunt.

44. The method of claim 42, wherein the inflating the balloon catheter further comprises inflating a single balloon.

45. The method of claim 44, wherein the balloon is longer than the covered center portion of the shunt.

46. The method of claim 44, wherein the covered center portion of the shunt restricts expansion of a center region of the balloon and thereby allows end regions of the balloon to expand to larger diameters than the center region.

47. The method of claim 44, wherein the single balloon is composed of a compliant material and a non-compliant band.

48. The method of claim 44, wherein end regions of the single balloon are comprised of material with different expansion properties than a middle region of the balloon.

49. The method of claim 42, wherein placing the delivery device into the artery and the vein of the patient comprises advancing a guidewire from the vein and into the artery.

50. The method of claim 49, wherein the guidewire is an RF guidewire.

51. The method of claim 49, wherein placing the delivery device into the artery and the vein of the patient comprises positioning a target sheath and directing the guidewire towards the target sheath.

52. The method of claim 51, wherein the target sheath comprises a target snare or target balloon.

53. The method of claim 37, wherein the first end and the second end of the shunt has an expanded diameter of about 4 mm.

54. The method of claim 53, wherein the covered center portion of the shunt has an expanded diameter of about 2 mm.

55. A method of creating a fistula in a patient, comprising:
advancing a wire from a vein of a patient into an artery of the patient;
advancing a delivery device over the wire and into both the artery and the vein of the patient;
inflating one or more balloons underneath a radially compressed shunt so as to radially flare a first end of the shunt within the artery and against a wall of the artery, and radially expanding a second end of the shunt within the vein and against a wall of the vein, while also longitudinally foreshortening the shunt; and,
establishing blood flow from the artery to the vein, through a covered center portion of the shunt and across a tissue gap.

56. A method of creating a fistula in a patient, comprising:
radially flaring a first end of a shunt within an artery and against a wall of the artery, and radially expanding a second end of the shunt within a vein and against a wall of the vein, while also longitudinally foreshortening the shunt; and,
establishing blood flow from the artery to the vein, through a covered center portion of the shunt and across a tissue gap.
